(19) United States
(12) Patent Application Publication
(10) Pub. No.: US 2020/0139011 A1
(43) Pub. Date: May 7, 2020

(54) EXTRACELLULAR MATRIX POUCH STRUCTURE AND USES THEREOF

(71) Applicant: AZIYO MED, LLC, Silver Spring, MD (US)

(72) Inventors: Minh VO, Silver Spring, MD (US); Andrew GREEN, Silver Spring, MD (US)

(21) Appl. No.: 16/667,841

(22) Filed: Oct. 29, 2019

Related U.S. Application Data
(60) Provisional application No. 62/754,935, filed on Nov. 2, 2018.

(51) Int. Cl. A61L 27/36

(52) U.S. Cl. CPC A61L 27/3633; A61N 1/3956; A61L 27/3683; A61L 27/3641

(57) ABSTRACT

The present invention provides an implantable medical product in a pouch structure, comprising an extracellular matrix (ECM) structure in sheet form. The pouch structure has an internal region and is configured to receive an electrical medical device therein. In particular, a larger pouch structure is designed to accommodate a medical device having a larger size, such as a subcutaneous implantable cardioverter-defibrillators (S-ICDs). In particular, lock-stitched seamed ECM sheets form a larger pouch structure. It is preferable that the stitches can securely hold the ECM sheets together by providing mechanical strength and durability to withstand the weight of the electronic medical device. The stitches securely hold the pouch structure together and does not fall apart during trimming or cutting of the pouch structure.

### CROSS-REFERENCE TO RELATED APPLICATION

This application claims the benefit of provisional U.S. patent application No. 62/754,935, filed Nov. 2, 2018, the disclosure of which is hereby incorporated by reference in its entirety.

### FIELD OF THE INVENTION

The present invention relates to an implantable medical product, methods and systems for generating a natural extracellular matrix pouch structure containing a medical device therein to be implanted in a patient, such as a pacemaker, defibrillator, or subcutaneous implantable cardioverter-defibrillators.

### BACKGROUND OF THE INVENTION

There are great needs to implant a variety of devices and systems for sensing, affecting, or performing patient's body functions, such as pacing devices, defibrillators, implantable access systems, monitors, stimulator, ventricular assist devices, pain pumps, infusion pumps and other systems to deliver energy or substances. Some treatments of medical conditions involve implantation of medical devices or insertion of medical instruments into a body, such as the implantation or deployment of heart valves to regulate the flow of blood through cardiovascular vessels, or the implantation of pacemakers to control abnormal heart rhythms. Cardiac pacing by an artificial pacemaker delivers a stimulus to the heart when its own natural pacemaker and/or conduction system fails to provide synchronized atrial and ventricular contractions at desirable rates and intervals. Such pacing provides relief from symptoms and even life support for hundreds of thousands of patients. Cardiac pacing is usually performed by a pulse generator implanted subcutaneously or sub-muscularly in or near a patient's pectoral or abdominal region.

Several cardiovascular implants are formed from natural tissue, such as heart valves disclosed in U.S. Pat. No. 6,719,788 (Apr. 13, 2004, James L. Cox) and U.S. Pat. No. 5,480,424 (Jan. 2, 1996, James L. Cox). The disclosed bioprostheses can, however, be affected by gradual calcification, which can, and in many instances will, lead to the eventual stiffening and tearing of the implant. Some non-bioprosthetic implants are fabricated from various metals and polymeric materials, and other exotic materials, such as pyrolytic carbon-coated graphite. For example, pacemakers, defibrillators, leads, and other similar cardiovascular implants are often fabricated from Ni—Co—Cr alloy, Co—Cr—Mo alloy, titanium, and Ti-6Al-4V alloy, stainless steel, and various biocompatible polymeric materials. Artificial heart valves are often fabricated from various combinations of nylon, silicone, titanium, Teflon™, polyacetal, graphite and pyrolytic carbon. Artificial hearts and ventricular assist devices are often fabricated from various combinations of stainless steel, cobalt alloy, titanium, Ti-6Al-4V alloy, carbon fiber reinforced composites, polyurethanes, Biolon™, Hemothane™, Dacron™, polysulfone, and other thermoplastics. Catheters and guide wires are often fabricated from Co—Ni or stainless steel wire. In many instances, the wire is encased in a polymeric material.

As is well known in the art, several major problems are often encountered when a medical device fabricated from one of the aforementioned materials is implanted in the body. A major problem that is often encountered after implantation of such a device in the body is inflammation of surrounding tissue. Another major problem is the high incidence of infection. A further problem that is often encountered after implantation of the medical device in the body is the formation of blood clots (thrombogenesis). One additional problem that is also often encountered is the degradation, e.g., corrosion, of medical device leads and, thereby, premature failure of the device after implantation in the body.

Most medical devices are designed to be implanted in the body for an extended period of time. However, when a harsh biological response (or premature failure of the device) is encountered after implantation, it is often necessary to remove the device through a secondary surgical procedure, which can, and in many instances will, result in undesirable pain and discomfort to the patient, and possibly additional trauma to the adjacent tissue. In addition to the pain and discomfort, the patient must be subjected to an additional time consuming and complicated surgical procedure with the attendant risks of surgery.

The implantations of medical devices have unique biocompatibility requirements to ensure that the device is not rejected or triggering adverse thrombogenic responses. There is thus a need to provide medical devices that are configured for implantation in the body, and substantially reduce or eliminate the harsh biological responses associated with conventional implanted medical devices, including inflammation, infection and thrombogenesis.

WO 2014/046741 A1 (Mar. 27, 2014, Robert G. Matheny) and US 2017/0304507 A1 (Oct. 26, 2017, Robert G. Matheny) disclose an encasement structure comprising a pouch formed from at least one sheet of extracellular matrix material having multiple plies or layers, with the pouch configured to have an internal region for receiving an electrical medical device therein. WO 2012/018680 A1 (Feb. 9, 2012, Joseph B. Horn et al.) discloses a medical implant that incorporates an electronic or medical device, such as a pocket-like implant having an interior space, which is implantable in a patient with the electronic medical device positioned in the interior space.

Despite the prior efforts of providing implantable medical devices to satisfy the biocompatibility requirements, there is still a need to provide various pouch structures at relatively low cost that securely hold medical devices therein when implanted in the body. In particular, there is a new need to configure specially designed pouch structures to accommodate various medical devices, such as medical devices which have different shapes, sharp edges, connecting conduits, wires, tubes, heavier weights or larger sizes.

### SUMMARY OF THE INVENTION

The present invention now provides methods for preparing pre-grafts and tissue grafts in the form of pouch structures and to the pouch structures formed by the methods.

In one aspect, the invention provides a method for making an implantable medical product of extracellular matrix (ECM) material in sheet form and having a relatively large size by following a number of preparation steps. First, a pair of first and second generally rectangular ECM sheets are provided, each including at least two ECM plies or layers and having top, bottom and two side edges. Next, a first side edge of the first sheet is overlapped onto a first side edge of the second sheet, followed by the joining together of the overlapped side edges by a first lock-stitched seam. Then, the pair of first and second generally rectangular ECM sheets having the joined overlapped side edges are superimposed upon each other, and are joined together by a peripheral lock-stitch seam that is provided along and that joins together the bottom edges of the sheets and that extends along at least a portion of each of the adjacent second side edges of the sheets. This forms a pouch structure having an internal region or cavity and an opening, with the top edges of the sheets not joined together to form the opening.

The first and second ECM sheets are preferably provided by trimming larger ECM sheets to remove defects. Also, the first and second ECM sheets having the overlapped side edges and first lock-stitched seam are die cut to provide rounded corners and a plurality of holes prior to joining the pair of first and second ECM sheets by the peripheral lock-stitched seam. Advantageously, the first lock-stitched seam extends substantially completely along the overlapped edges of the sheets and the peripheral lock-stitched seam extends substantially completely along one of the second side edges of the sheets and about half-way along the other of the second side edges of the sheets. This facilitates manipulation of the opening to allow the medical device to be placed into the cavity of the pouch structure.

The method further comprises inserting an electrical medical device into the internal region or cavity of the pouch structure, wherein the medical device is a pacemaker, defibrillator, synthetic heart valve, ventricular assist device, artificial heart, physiological sensor, catheter, or an associated component thereof. These devices can be of larger size than usual as the pouch is also of a relatively large size. After inserting the electrical device therein, the opening of the pouch structure can be closed by stitching or stapling.

If desired, a composition that is a pharmacological agent can be introduced into the internal region or cavity of the pouch structure. Preferably, the pharmacological agent is or includes an antibiotic agent which is added prior to inserting the medical device into the pouch structure.

It has been found that the lock-stitch seams provide dimensional stability to the pouch structure, especially when it is hydrated. Additionally, folding the edges of any or all of the sheets prior to stitching to provide a double thickness of the ECM sheets being stitched together provides further structural integrity. And the pouch structure may be trimmed without loss of closure because the lock-stitched seams do not unravel when cut or trimmed.

In another aspect, the invention provides the medical product that is prepared by the methods described herein. Such a medical product is implantable and generally comprises a pouch structure of an extracellular matrix (ECM) structure in sheet form as described herein. This the pouch structure comprises an internal region or cavity and an opening, and is configured and sized to receive, encase and retain an electrical medical device therein and to allow such device to be inserted into the internal region or cavity of the pouch structure. The pouch structure comprises a pair of first and second generally rectangular ECM sheets each including at least two ECM plies or layers and having top, bottom and two side edges, wherein a first side edge of the first sheet overlaps a first side edge of the second sheet with the overlapped side edges joined together by a first lock-stitched seam. The pair of first and second generally rectangular ECM sheets are superimposed upon each other and joined together with a peripheral lock-stitch seam that is provided along and that joins together the bottom edges of the sheets and that extends along at least a portion of each of the adjacent second side edges of the sheets. As noted above, the top edges of the sheets are not joined together to form the opening of the pouch structure and are configured for closing by stitching or stapling after receiving the device.

The first and second ECM sheets each preferably have a rounded corner away located within the peripheral lock-stitched seam, with the first lock-stitched seam extending substantially completely along the overlapped edges of the sheets and the peripheral lock-stitched seam extending substantially completely along one of the second side edges of the sheets and about half-way along the other the second side edges to provide an opening for a lead conduit of the medical device. Also, each ECM sheet typically includes a plurality of holes in a spaced pattern.

The invention also relates to a medical product that is the combination of the pouch structure disclosed herein an electrical medical device wherein the internal region or cavity of the pouch structure contains the electrical medical device therein. The medical device is typically a pacemaker, defibrillator, synthetic heart valve, ventricular assist device, artificial heart, physiological sensor, catheter, or an associated component thereof, and can be of larger size such as a subcutaneous implantable cardioverter-defibrillator or programmable pump due to the larger size of the pouch structure that is provided.

As noted, the internal region or cavity of the pouch structure may contain a composition that is a pharmacological agent, which is or includes an antibiotic agent. And for optimum strength, the edges of any or all of the sheets are folded to provide a double thickness of the ECM sheets joined by the lock-stitched seams.

### BRIEF DESCRIPTION OF THE DRAWING FIGURES

The nature and various advantages of the present invention will become more apparent upon consideration of the following detailed description, taken in conjunction with the accompanying drawings, and in which:

FIG. 1 is a perspective view of one embodiment of assembling a pouch structure by overlapping two rectangular ECM sheets at one of the four side-edges and joining two ECM sheets at the overlapped area to produce one larger seamed-ECM sheet.

FIG. 2 is a perspective view of one embodiment of assembling a pouch structure by overlapping two larger seamed-ECM sheets and joining them together by stitching the outer peripheries of the seamed-ECM sheets.

FIG. 3 is a perspective view of an assembled pouch structure.

### DETAILED DESCRIPTION OF THE INVENTION

Throughout this description, the preferred embodiments and examples provided herein should be considered as exemplary, rather than as limitations of the present invention.

The present invention provides an implantable medical product that includes a bioremodelable pouch structure comprising an extracellular matrix (ECM) material in sheet form, wherein the pouch structure has an internal region and is configured to receive an electrical medical device therein. In particular, a larger pouch structure is designed to accommodate a medical device having a larger size, such as a subcutaneous implantable cardioverter-defibrillator. The larger pouch structure of the present invention creates a natural regenerative implantation environment by preserving defibrillation threshold vectors with a stable shock impedance. The pouch structure of the present invention can thus provide a long-term healthy pocket and vector stabilization.

In particular, two smaller size rectangular ECM sheets are joined along an edge with a lock-stitched seam to form a larger ECM sheet. These larger sheets are joined together by lock-stitching along a portion of the periphery of their edges, to form a larger pouch structure. It is preferable that the stitches can securely hold the ECM sheets together by providing mechanical strength and durability to withstand the weight of the electronic medical device. In particular, these lock stitches can securely hold the pouch structure together and do not fall apart during cutting or trimming of the pouch structure.

The pouch structure of the present invention can securely hold medical devices when implanted in the body. In particular, the pouch structure of the present invention is configured specifically to accommodate various medical devices, such as medical devices which have different shapes, sharp edges, connecting conduits, wires, tubes, heavier weights or larger sizes.

The ECM structure contains acellular ECM material from a decellularized mammalian tissue source. This decellularized mammalian tissue source is typically small intestine submucosa (SIS), urinary bladder submucosa, stomach submucosa, epithelium of mesodermal origin i.e. mesothelial tissue, dermal extracellular matrix, subcutaneous extracellular matrix, gastrointestinal extracellular matrix i.e., large and small intestines, tissue surrounding growing bone, placental extracellular matrix, omentum extracellular matrix, cardiac extracellular matrix e.g., pericardium and/or myocardium, kidney extracellular matrix, pancreas extracellular matrix, lung extracellular matrix, urinary basement membrane, liver basement membrane, or various combinations thereof. The ECM material can also comprise collagen from mammalian sources. The ECM material can be derived from various mammalian tissue sources and methods for preparing same, such as disclosed in U.S. Pat. Nos. 9,700,654, 9,333,277, 8,758,448, 7,550,004, 7,244,444, 6,379,710, 6,358,284, 6,206,931, 5,733,337 and 4,902,508, which are incorporated by reference herein in their entirety.

The pouch structure of the present invention includes an internal region or cavity and an opening, wherein the pouch structure is configured and sized to receive, encase and retain an electrical medical device therein and to allow such device to be inserted into the internal region or cavity of the pouch structure. The pouch structure is formed from a pair of first and second ECM sheets, with each ECM sheet having edges or peripheries, wherein the edges of the ECM sheets are joined together except for the edges that form the opening to provide the internal region or cavity between the sheets. Certain edges of the ECM sheets of the pouch structure are joined together by lock-stitching so that the pouches can be trimmed or reduced in size without compromising the strength of the stitching that joins the sheets together. It is preferable that the opening is configured for closing by stitching or stapling those edges after receiving the device.

The electrical medical device to be encased inside the pouch structure of the present invention includes any of a pacemaker, defibrillator, synthetic heart valve, ventricular assist device, artificial heart, physiological sensor, catheter, subcutaneous implantable cardioverter-defibrillator (S-ICD), or an associated component thereof. The larger pouch structures of the present invention can accommodate larger devices such as the S-ICDs. Other medical devices that can be accommodated by the larger pouch structures of the present invention includes pumps such as the Prometra® programmable pump for use with Intrathecal Catheters, or Medtronic SynchroMed® II programmable pumps. These devices have thicknesses between 15 and 30 mm with diameters of about 65 to 95 mm (6.5 to 9.5 cm). Such pump devices can be retained in the larger pouch structures of the present invention. After the insertion of the medical device, all open edges of the pouch structure are closed by stitches at small intervals with at least one protruding object, such as a lead conduit of the medical device, extending out of the pouch structure between the edges and stitching, typically along one of the sides of the pouch structure. In one embodiment, a pouch structure is configured to encase a medical device which comprises at least one lead conduit, more preferably, a plurality of lead conduits, wherein the pouch structure can encase the medical device with the lead conduits passing through a side seam.

By joining two smaller rectangular ECM sheets together, larger pouch dimensions can be obtained. The dimensions of the pouch structure are variable depending on the size, shape and weight of the medical device to be retained therein. In one embodiment, the pouch structure has the dimensions of 5.4 cm width by 5.0 cm height, 6.9 cm width by 6.5 cm height, 6.9 cm width by 8.0 cm height, 6.9 cm width by 9.5 cm height, or 10.8 cm width by 8.9 cm height.

In one embodiment, larger pouch structures are preferred, having dimensions of approximately 10.8 cm by 8.9 cm, as it is designed to accommodate a medical device having a larger size, such as a subcutaneous implantable cardioverter-defibrillator (S-ICD). Two rectangular ECM sheets are used to assemble a larger seamed-ECM sheet, wherein each of the rectangular ECM sheets has four side-edges. Two rectangular ECM sheets are joined together at one of the side-edges of the rectangular shape to produce one larger seamed-ECM sheet. Two rectangular ECM sheets having four side-edges are piled together at one side-edge to provide an overlapped area of the sheets, wherein the overlapped side-edges of the ECM sheets are sewed together at the overlapped area by lock-stitching.

Some of the smaller size pouch structures can be prepare by lock stitching the peripheral edge portions of two single sheets, but it is preferred for a pair of first and second sheets to joined together prior to forming the pouch as the center seam has been found to add support to the pouch structure such that only two layers or plies of ECM material are sufficient to retain the medical device therein. This reduces the cost of the pouch structure by at least 30% compared to pouch structures formed with 3 or 4 plies of ECM materials in the sheets, even though additional sewing is needed. And as the sewing is conducted on a machine, preparation time is conserved.

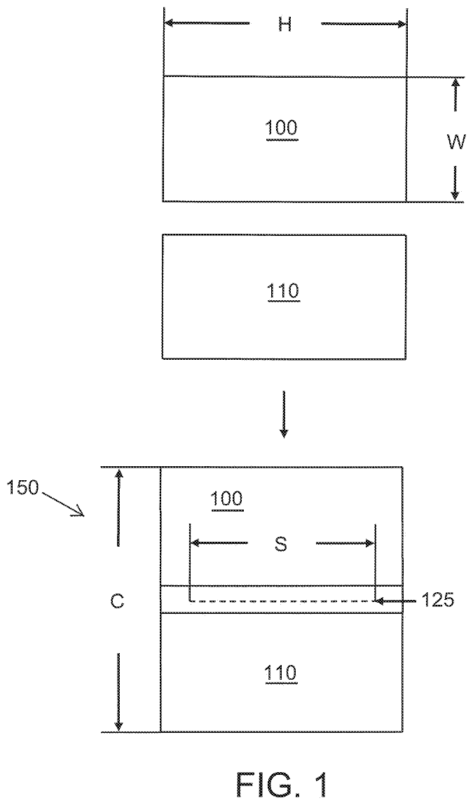

FIG. 1 is a perspective view of one embodiment of preparing a larger pouch structure using rectangular ECM sheets 100, 110, wherein each of the rectangular ECM sheets has four side-edges. The two pieces of rectangular ECM sheets 100, 110 are provided by cutting larger pieces to remove defects or other inhomogeneities. As shown in FIG. 1 at 150, the sheets are placed at one of their side edges to provide an overlapped area. A center-seam 120 is made on the overlapped area to join the edges of the two rectangular ECM sheets together, therefore producing one larger ECM sheet having a central, lock-stitched seam. In one embodiment, each of the rectangular ECM sheets has at least two layers or plies of ECM material. If desired, multiple layers of 3 or more plies can be used although this increases the cost of the product.

Typical height H and width W of the individual first and second sheets range from 5 to 12 cm for H and 4 to 8 cm for W. A preferred size is approximately 10 cm by 6.3 cm. Typical widths C of the combined sheets range from 8 to 16 cm while the height would be about the same as that of the individual sheets. A preferred size of the combined ECM sheet then would be approximately 11.3 cm by 10 cm as the edges overlap by about 1.3 cm. The center seam has a length S of about 6 cm. This represents substantially complete coverage of about 95% of the length of the sheet. Generally, the center seam length S should be at least 75% to as close to 100% of the length of the sheet while the overlap of the edges can be as much as 1.5 cm.

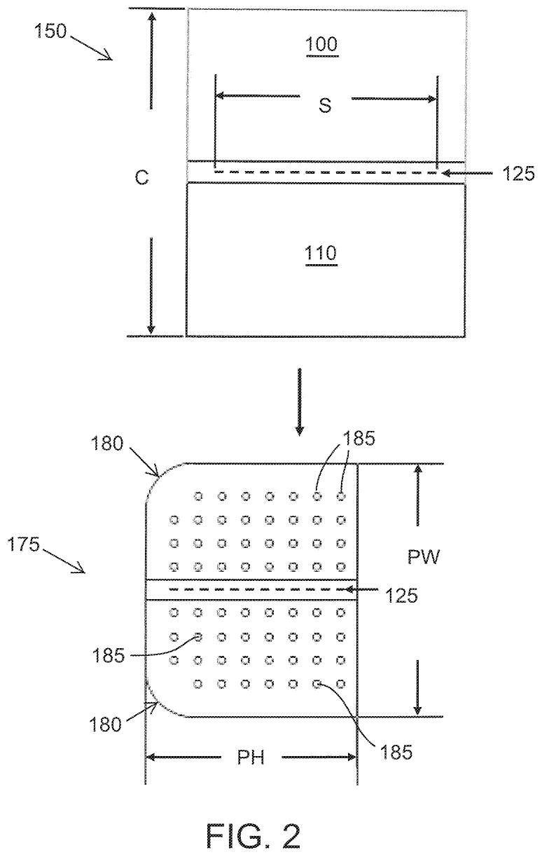

FIG. 2 is a perspective view of one embodiment of assembling a pouch structure by superimposing two edge seamed-ECM sheets prior to joining them together by stitching certain portions of the peripheries of the sheet edges. The two rectangular seamed sheets 150 are die cut to provide modified sheets 175 having rounded corners 180 and a plurality of holes 185. The pouch structure width PW ranges from 9 to 15 cm while the pouch structure height PH ranges from 7 to 10 cm. The pouch structure has typical dimensions of at approximately 11 cm by 9 cm.

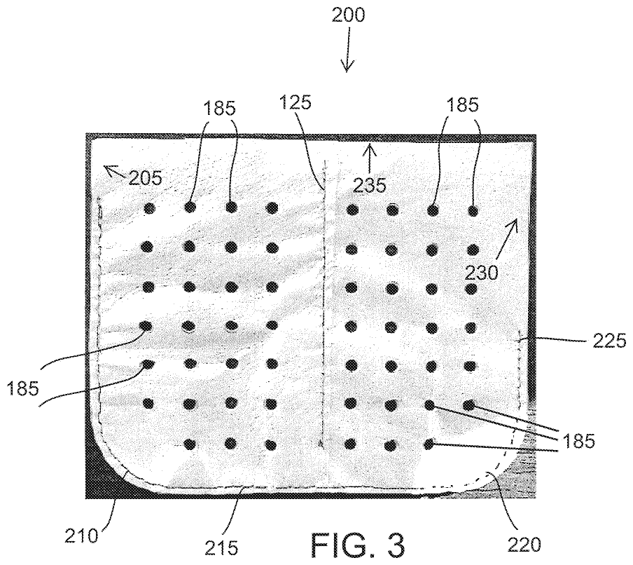

FIG. 3 is a perspective view of a lock-stitched final pouch structure 200. The peripheral lock stitching starts at an upper portion of a side edge 205 and continues to and around one round corner 210 then across the bottom edge 215 of the sheets to the other round corner 220 to a termination point around the middle of the opposite side edge 225. The upper half 230 of the opposite side edge and the top edges 235 are not stitched and remain open. The top edges can be separated to insert the medical device with the lead conduits of the device passing through the upper half 230 of the side edge for allowing exit of the lead conduits of the device. A surgeon can then stitch or staple the opening to secure the device therein with the leads passing out of the pouch structure.

In one embodiment, two pieces of the larger seamed-ECM sheets 150 are overlapped to form a pouch structure by stitching the outer periphery of the seamed-ECM sheets to create a side-seam with an open seam allowance as shown in FIG. 3 as 235. A seam allowance is the area between the edge of the seamed-ECM sheets and the stitching line on two or more pieces of the seamed-ECM sheets being stitched together. A seam is the join where two or more layers of materials are held together with stitches. In one embodiment, a side-seam is made on a periphery of the seamed-ECM sheets with folded edges with a seam allowance.

In a preferred embodiment, a pouch structure comprises two rectangular seamed-ECM sheets 150, wherein each of the seamed-ECM sheets has four outer side-peripheries, wherein three side-peripheries of the seamed-ECM sheets are joined together by stitching. An opening located in the fourth side-periphery of the seamed-ECM sheets of the pouch structure is intended for inserting the medical device into the interior space of the pouch structure. It is preferable that the fourth periphery is the longer periphery of the rectangular seamed-ECM sheets of the pouch structure.

In a preferred embodiment, a pouch structure comprises two rectangular seamed-ECM sheets, wherein each of the seamed-first and second ECM sheets has four side-peripheries, wherein two adjacent side-peripheries of each of the seamed-ECM sheet are substantially completely joined together by stitching, wherein the third side-periphery of each of the seamed-ECM sheet is joined together by partial stitching, such as along about half of the length of the edge. A partial opening located in the third side-periphery of the seamed-ECM sheets of the pouch structure is intended for accommodating any protruding components of the medical device, such as conduits, electrical wires, or tubes. An opening located in the fourth side-periphery of the seamed-ECM sheets of the pouch structure is intended for inserting the medical device into the interior space of the pouch structure. It is preferable that the fourth periphery is the longer periphery of the rectangular seamed-first and second ECM sheets.

When the edges or peripheries of the ECM sheets are joined together by stitching of thread or sutures, the stitching is made by a medical grade sewing machine of the various types that are used for sewing various polymer materials for use in medical gowns or drapes. The stitches are preferably made as lock-stitches in order to securely hold the ECM sheets together in the pouch structure. Such lock-stitching provides mechanical strength and durability to withstand the weight of the electronic medical device. It is particularly preferable that the stitches or sutures can securely hold the ECM sheets together, when the pouch structure is trimmed or sized. In other words, the stitches or sutures will not fall apart during trimming or sizing the pouch structure. In particular, the lock-stitch uses two threads, an upper and a lower thread, and are entwined together in the edges of the ECM sheets in order to be locked therein. It is preferable to use a sewing machine to generate the lock-stitches for best and repeatable results.

In a preferred embodiment, the ECM pouch structure is configured to encase an entire medical device. In some embodiments, the ECM pouch structures is also configured to encase at least a portion of the medical device electrical leads, generally through one of the seams of the pouch structure.

In some embodiments, the ECM pouch structure of the present invention includes at least one additional biologically active agent or composition, i.e. an agent that induces or modulates a physiological or biological process, or cellular activity, e.g., induces proliferation, and/or growth and/or regeneration of tissue, such as a growth factor, cell, or chitosan.

In some embodiments, the ECM pouch structure of the present invention includes at least one pharmacological agent or composition, i.e. an agent or composition that is capable of producing a desired biological effect in vivo, e.g., stimulation or suppression of apoptosis, stimulation or suppression of an immune response, etc. Suitable pharmacological agents and compositions include antibiotics, anti-viral agents, analgesics, and steroidal and non-steroidal anti-inflammatories. In some embodiments, the pharmacological agent or composition includes antibiotics, antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or anti-thrombogenic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, vasodilating agents, anti-inflammatory agent, or HMG-CoA reductase inhibitor. The antibiotics are most preferred due to the beneficial effect they provide.

The pharmacological agent or composition can also include chemotherapy agents, including antimetabolites, such as purine analogues, pyrimidine analogues and anti-folates, plant alkaloids, such as vincristine, vinblastine, vinorelbine, vindesine, podophyllotoxin, etoposide and teniposide, taxanes, such as paclitaxel and docetaxel, topoisomerase inhibitors, such as irinotecan, topotecan, amsacrine, etoposide, etoposide phosphate and teniposide, cytotoxic antibiotics, such as actinomyocin, bleomycin, plicamycin, mytomycin and anthracyclines, such as doxorubicin, daunorubicin, valrubicin, idarubicin, epirubicin, and antibody treatments, such as abciximab, adamlimumab, alamtuzumab, basiliximab, belimumab, bevacizumab, brentuximab vedotin, canakinumab, cetuximab, certolizumab pego, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumumab, ranibizumab, rituximab, tocilizumab (atlizumab), tositumomab or trastuzumab.

The pharmacological agent or composition can also include anti-inflammatory agents inclduing alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, or zomepirac sodium.

In some embodiments, the pharmacological agent comprises a statin, i.e. a HMG-CoA reductase inhibitor. Suitable statins include atorvastatin (Lipitor®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

The ECM material can be derived from basement membrane of mammalian tissue/organs, including urinary basement membrane (UBM), liver basement membrane (LBM), and amnion, chorion, allograft pericardium, allograft acellular dermis, amniotic membrane, Wharton's jelly, and combinations thereof. Additional sources of mammalian basement membrane include, without limitation, spleen, lymph nodes, salivary glands, prostate, pancreas and other secreting glands.

In one embodiment, the pouch structure of the present invention comprises an ECM structure in sheet form with joined edges or peripheries of multiple piled/stacked ECM sheets. Only the edges or peripheries of the ECM sheets of the pouch structure of the present invention are joined together, preferably by the lock stitching. When only the edges or peripheries of the plies of the ECM sheets are joined together by stitching, suturing or stapling, they minimize the damages on the ECM structure without altering the naturally occurring ECM bioscaffold.

Alternatively, the multiple piled/stacked ECM sheets can be laminated together to form an integral ECM sheet for processing according to the present methods.

In a preferred embodiment, the ECM material is obtained from small intestine submucosa (SIS) in sheet form. The ECM material obtained from porcine SIS is a decellularized matrix that serves as a bioscaffold to allow vascular ingrowth from adjacent tissues to deliver progenitor cells and nutrients to the matrix, which then differentiate into tissue-specific cells and structures. The ECM is gradually replaced as the patient's own cells by restoring the diseased or damaged site. During repair, the matrix is naturally degraded and resorbed, leaving remodeled functional tissue where damaged or injured tissue would normally be expected.

The ECM sheet of the pouch structure of the present invention is typically made of at least two ECM plies or layers. A greater number of ECM layers can be used but the advantage of combining lock-stitching with two layer ECM sheets allows the cost of the pouch material to be reduced without compromising on the strength and resiliency of the pouch structure. When more than one ECM layer is used to produce the ECM sheet, this provides the advantages of increased tensile and mechanical strengths. In a preferred embodiment, the ECM sheet has two ECM plies or layers, i.e., a two-layered sheet, wherein the ECM material is obtained from SIS as this provides the best combination of cost and performance.

Two or multiple ECM layers can be combined, overlapped, piled and fused together to form one ECM sheet by compressing the overlapping areas under dehydrating conditions, or using conventional methods to combine ECM materials, such as lamination. In one embodiment, the ECM sheet comprises multiple layers of ECM material which are decellularized, non-crosslinked, and lyophilized.

U.S. Pat. No. 8,128,708 (Mar. 6, 2012, Michael C. Hiles et al.) discloses a purified form of a submucosa tissue that has the capability of being shape formed or shape configured to confer some shape memory and shape configuration to the implant. The purified submucosa can be prepared in sheet form and packaged to permit sterility or maintain sterility of the submucosa. The submucosa is shown to exhibit more remodeling, regrowth, and regeneration of tissue upon implant compared to using collagen. It has been shown that submucosal tissue is absorbed by the patient and thus the patient does not require post-implantation procedures to remove the implant. The submucosal tissue has been shown to have favorable immune response that leads to an accommodation of the submucosal implant versus a rejection based response. The content of U.S. Pat. No. 8,128,708 is incorporated herein by reference in its entirety as this type of tissue provides additional benefits when prepared in a pouch structure as disclosed herein.

The larger pouch structure of the present invention, such as the dimension of approximately 10.8 cm by 8.9 cm, creates a natural regenerative implantation environment by preserving defibrillation threshold vectors with a stable shock impedance, which are challenging complications for patients undergoing implantation of S-ICDs. The pouch structure of the present invention is large enough to secure the S-ICD, which is critical for the potential to achieve effective defibrillation. In particular, the pouch structure of the present invention can provide a long-term healthy pocket and vector stabilization.

The pouch structure of the present invention provides unexpected synergistic advantages by combining the features of having a naturally occurring ECM bioscaffold and having the required mechanical strength to withhold the weight of the electronic medical device. Only the edges or peripheries of the ECM sheets of the pouch structure of the present invention are stitched or stapled together, such as by stitching unconnected edge portions at small intervals, the pouch structure of the present invention provides the benefit of having a naturally occurring ECM bioscaffold with minimal alteration by preserving the properties, structures and characteristics of naturally occurring ECM. The benefit of having a naturally occurring ECM bioscaffold is to promote tissue regeneration without scarring, since the altered ECM can cause inflammation, scar, or clogged blood flow for the invention of wound healing. The pouch structure provides the benefits of substantially reducing or eliminating the harsh biological responses associated with conventional implanted medical devices, including inflammation, infection and thrombogenesis, therefore substantially enhancing biocompatibility and hemocompatibility.

In addition, the pouch structure of the present invention provides the benefit of having the required mechanical strength to securely hold an electronic medical device, such as cardiac implantable electronic devices (CIEDs) including pacemakers or defibrillators, to be implanted in the body. The unique design of the pouch structure of the present invention facilitates the creation of a stabilized environment to enhance patient comfort by reducing device migration and erosion. The stabilized environment can also facilitate the removal of the implanted medical device during exchange and revision.

The feature of having larger size ECM sheets with lock-stitched edges offers the advantages of providing the right amount of mechanical strength to retain the right shape of the pouch structure with the desired tensile strength without altering the shape of the pouch. The mechanical strength of the pouch structure creates a stabilized environment that enhances patient comfort and reduces device migration. The pouch structure of the present invention conforms to the implantable device and remodels into a defined, vascularized pocket that supports and reinforces, while reducing migration of CIEDs. After the implantation, the pouch structure can be remodeled into systemically connected neovascularized tissue.

In a preferred embodiment, the pouch structure of the present invention does not have sharp edges, since the periphery of the corner of the rectangular shape of the pouch structure is curved and round. In an embodiment, stitching or suturing is used to join the ECM sheets, the ECM sheets are stitched together by placing the side-seams within the ECM sheet without covering the edges of the ECM sheets. The soft and supple edges of the pouch structure enhance patient comfort, facilitate implantation, and simplify device removal for exchanges or revisions.

Tissue regeneration is the ability to make tissue regrow, such as to regrow an organ by itself or to reform tissue without scarring. Healing a wound is the ability of the tissue to heal preferably without scarring or with very minimal scarring. The edges of the ECM sheets are stitched together to provide the tensile strength to withhold the weight of the electronic medical device. In particular, with regard to the use of first and second multilayer ECM sheets, since a cavity is formed using the multilayer ECM sheets or sheet portions with joined edges, the pouch structure of the present invention provides a mechanical strength to securely hold the electrical medical device in place, additionally providing durability, tensile strength, flexibility and softness.

One important advantage of the present invention is the use of sheets that include plural layers of ECM material from the same or different mammalian tissues which have various attributes. The synergistic attributes of the ECM layers of the pouch structure of the present invention provide tensile strength to support newly forming tissue and tissue regeneration by providing mechanical strength to hold the medical device with multiple layers of ECM sheets which are stitched together at the edges of the encasement layers. These features provide benefits in holding the electrical medical device properly, which are particularly beneficial under the condition that the electrical medical devices have different shapes or contain rectangular edges.

The pouch structure of the present invention provides several synergistic advantages. The pouch structure of the present invention provides an ECM encasement structure in sheet form, which is configured to encase a medical device therein and can effectively improves biological functions by promoting tissue regeneration, modulated healing of adjacent tissue or growth of new tissue when implanted in patient. In addition, the pouch structure of the present invention is configured to encase a medical device therein and that substantially reduce or eliminate the harsh biological responses associated with conventional implanted medical devices, including inflammation, infection and thrombogenesis, when implanted in the body. The pouch structure of the present invention is configured to encase a medical device therein, and effectively improve biological functions and/or promote modulated healing of adjacent tissue and the growth of new tissue when implanted in the body. The pouch structure of the present invention is configured to encase a medical device therein and administer one or more pharmacological or therapeutic agents to a subject when implanted in his/her body. The pouch structure of the present invention is configured for insertion or implantation in the body and exhibit enhanced biocompatibility and hemocompatibility when inserted or implanted therein.

Modulated healing is referring to a process involving different cascades or sequences of naturally occurring tissue repair in response to localized tissue damage or injury, substantially reducing their inflammatory effect. Modulated healing includes many different biologic processes, including epithelial growth, fibrin deposition, platelet activation and attachment, inhibition, proliferation and/or differentiation, connective fibrous tissue production and function, angiogenesis, and several stages of acute and/or chronic inflammation, and their interplay with each other.

It is to be understood that additional embodiments of the present invention described herein may be contemplated by one of ordinary skill in the art and that the scope of the present invention is not limited to the embodiments disclosed. While specific embodiments of the present invention have been illustrated and described, numerous modifications come to mind without significantly departing from the spirit of the invention, and the scope of protection is only limited by the scope of the accompanying claims.

What is claimed is:

1. A method for making an implantable medical product that includes extracellular matrix (ECM) material in sheet form, which comprises:
providing a pair of first and second generally rectangular ECM sheets each including at least two ECM plies or layers and having top, bottom and two side edges;
overlapping a first side edge of the first sheet onto a first side edge of the second sheet;
joining together the overlapped side edges by a first lock-stitched seam;
superimposing the pair of first and second generally rectangular ECM sheets having the joined overlapped side edges;
joining together the pair of first and second generally rectangular ECM sheets by a peripheral lock-stitch seam that is provided along and that joins together the bottom edges of the sheets and that extends along at least a portion of each of the adjacent second side edges of the sheets to form a pouch structure that includes an internal region or cavity and an opening;
wherein the top edges of the sheets are not joined together to form the opening.

2. The method of claim 1, wherein the first and second ECM sheets are provided by trimming larger ECM sheets to remove defects, with the ECM material comprising acellular ECM from a decellularized mammalian tissue source, with the decellularized mammalian tissue source being small intestine submucosa (SIS), urinary bladder submucosa, stomach submucosa, epithelium of mesodermal origin, dermal extracellular matrix, subcutaneous extracellular matrix, gastrointestinal extracellular matrix, tissue surrounding growing bone, placental extracellular matrix, omentum extracellular matrix, cardiac extracellular matrix, kidney extracellular matrix, pancreas extracellular matrix, lung extracellular matrix, urinary basement membrane, liver basement membrane, or combinations thereof.

3. The method of claim 1, which further comprises, die cutting the first and second ECM sheets having the overlapped side edges and first lock-stitched seam to provide rounded corners and a plurality of holes prior to joining the pair of first and second ECM sheets by the peripheral lock-stitched seam.

4. The method of claim 1, wherein the first lock-stitched seam extends substantially completely along the overlapped edges of the sheets and the peripheral lock-stitched seam extends substantially completely along one of the second side edges of the sheets and about half-way along the other of the second side edges of the sheets.

5. The method of claim 1, which further comprises inserting an electrical medical device into the internal region or cavity of the pouch structure.

6. The method of claim 5, wherein the medical device is a subcutaneous implantable cardioverter-defibrillator, a pump, a pacemaker, synthetic heart valve, ventricular assist device, artificial heart, physiological sensor, catheter, or an associated component thereof.

7. The method of claim 5, which further comprises closing the opening of the pouch structure by stitching or stapling after inserting the electrical device therein.

8. The method of claim 1, which further comprises providing a composition that is a pharmacological agent into the internal region or cavity of the pouch structure.

9. The method of claim 8, wherein the pharmacological agent is or includes an antibiotic agent which is added prior to inserting the medical device into the pouch structure.

10. The method of claim 1, which further comprises folding the edges of any or all of the sheets prior to stitching to provide a double thickness of the ECM sheets being stitched together.

11. A pouch product prepared by the method of any one of claims 1-10.

12. An implantable medical product, comprising:
a pouch structure comprising extracellular matrix (ECM) material in sheet form, wherein the ECM material comprises acellular ECM from a decellularized mammalian tissue source, with the decellularized mammalian tissue source being small intestine submucosa (SIS), urinary bladder submucosa, stomach submucosa, epithelium of mesodermal origin, dermal extracellular matrix, subcutaneous extracellular matrix, gastrointestinal extracellular matrix, tissue surrounding growing bone, placental extracellular matrix, omentum extracellular matrix, cardiac extracellular matrix, kidney extracellular matrix, pancreas extracellular matrix, lung extracellular matrix, urinary basement membrane, liver basement membrane, or combinations thereof,
wherein the pouch structure comprises an internal region or cavity and an opening, and is configured and sized to receive, encase and retain an electrical medical device therein and to allow such device to be inserted into the internal region or cavity of the pouch structure;
wherein the pouch structure comprises a pair of first and second generally rectangular ECM sheets each including at least two ECM plies or layers and having top, bottom and two side edges, wherein a first side edge of the first sheet overlaps a first side edge of the second sheet with the overlapped side edges joined together by a first lock-stitched seam, wherein the pair of first and second generally rectangular ECM sheets are superimposed upon each other and joined together with a peripheral lock-stitch seam that is provided along and that joins together the bottom edges of the sheets and that extends along at least a portion of each of the adjacent second side edges of the sheets; and
wherein the top edges of the sheets are not joined together to form the opening of the pouch structure and are configured for closing by stitching or stapling after receiving the device.

13. The implantable medical product of claim 12, wherein the first and second ECM sheets each have a rounded corner away located within the peripheral lock-stitched seam.

14. The implantable medical product of claim 12, wherein the first lock-stitched seam extends substantially completely along the overlapped edges of the sheets and the peripheral lock-stitched seam extends substantially completely along one of the second side edges of the sheets and about half-way along the other of the second side edges of the sheets to provide an opening for a lead conduit of the medical device.

15. The implantable medical product of claim 12, wherein each ECM sheet includes a plurality of holes in a spaced pattern.

16. The implantable medical product of claim 12, in combination with an electrical medical device wherein the internal region or cavity of the pouch structure contains the electrical medical device therein.

17. The implantable medical product of claim 16, wherein the medical device is a subcutaneous implantable cardioverter-defibrillator, a pump, a pacemaker, a synthetic heart valve, ventricular assist device, artificial heart, physiological sensor, catheter, or an associated component thereof.

18. The implantable medical product of claim 12, wherein the internal region or cavity of the pouch structure contains a composition that is a pharmacological agent.

19. The implantable medical product of claim 18, wherein the pharmacological agent is or includes an antibiotic agent.

20. The implantable medical product of claim 12 wherein the edges of any or all of the sheets are folded to provide a double thickness of the ECM sheets joined by the lock-stitched seams.
